(19) United States
(12) Patent Application Publication
(10) Pub. No.: US 2010/0331618 A1
(43) Pub. Date: Dec. 30, 2010

(54) Endoscope shaft frame member with wavy slot

(75) Inventor: Nison Galperin, Fairfield, CT (US)

Correspondence Address: HARRINGTON & SMITH, 4 RESEARCH DRIVE, Suite 202, SHELTON, CT, 06484-6212, US

(73) Assignee: Gyrus ACMI, Inc.

(21) Appl. No.: 12/456,986

(22) Filed: Jun. 24, 2009

(51) Int. Cl. A61B 1/00; B23P 11/00

(52) U.S. Cl. 600/101; 29/428

(57) ABSTRACT

An endoscope shaft frame member including a general tube shape with a section having a general helical shaped slot along a length of the section. The slot includes a general wavy shape along a length of the slot.

### BACKGROUND OF THE INVENTION

1. Field of the Invention

The invention relates to an endoscope and, more particularly, to an endoscope shaft frame member.

2. Brief Description of Prior Developments

U.S. Pat. No. 6,485,411 B1, which is hereby incorporated by reference in its entirety, discloses an endoscope shaft with a superelastic alloy spiral frame. The design has good resilient properties, but it has good torque stability in only the direction of the spiral cut.

An endoscope shaft is known which uses two spiral frame members; one with a left hand spiral and one with a right hand spiral. The two spiral frame members overly each other for torque stability, but do not have variable pitch and has a larger outer diameter. The two spiral frame member design also does not have good resilient properties.

Another design with an orthogonal cut slotted tube is know which is manufactured by Storz. However, although the design has good torque stability, the slotted tube has stress and fatigue problems, and not good resilient properties.

There is a desire for an endoscope shaft frame member which has good resilient properties, good stress and fatigue properties, and good torque stability in more than one direction, but without increasing the outer diameter size of the endoscope shaft.

### SUMMARY

The following summary is merely intended to be exemplary. The summary is not intended to limit the scope of the claimed invention.

In accordance with one aspect of the invention, an endoscope shaft frame member is provided including a general tube shape with a section having a generally helical shaped slot along a length of the section. The slot includes a general wavy shape along a length of the slot.

In accordance with another aspect of the invention, an endoscope shaft frame member is provided comprising a general tube shape with a section having a general helical shaped slot with a substantially uniform width between opposing surfaces of the section at the slot. The slot is non-straight along its general helical shape at the section.

In accordance with another aspect of the invention, a method is provided comprising providing a general tube shaped member; and forming a general helical shaped slot along a section of the tube shaped member. The general helical slot comprises a general wavy shape along its length.

### BRIEF DESCRIPTION OF THE DRAWINGS

The foregoing aspects and other features of the invention are explained in the following description, taken in connection with the accompanying drawings, wherein:

FIG. 1 is a side view of an endoscope comprising features of the invention;

FIG. 2 is a cross sectional view of the shaft of the endoscope shown in FIG. 1;

FIG. 3 is a perspective view of a frame member of the shaft shown in FIG. 2;

FIGS. 4-5 are enlarged views of the frame member shown in FIG. 3;

FIG. 6 shows the frame member of in FIGS. 3-5 being axially bent;

FIG. 7 is shows the frame member of in FIGS. 3-5 being axially twisted; and

FIGS. 8-10 show alternate embodiments of the frame member.

### DETAILED DESCRIPTION OF EMBODIMENTS

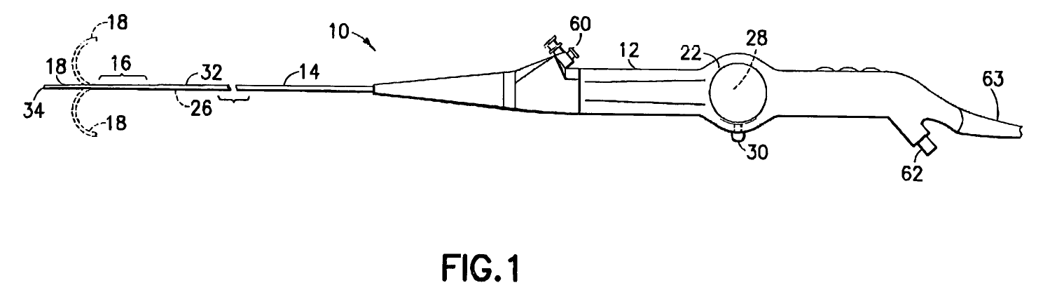

Referring to FIG. 1, there is shown a side view of an endoscope 10 incorporating features of the invention. Although the invention will be described with reference to the example embodiments shown in the drawings, it should be understood that the invention can be embodied in many alternate forms of embodiments. In addition, any suitable size, shape or type of elements or materials could be used.

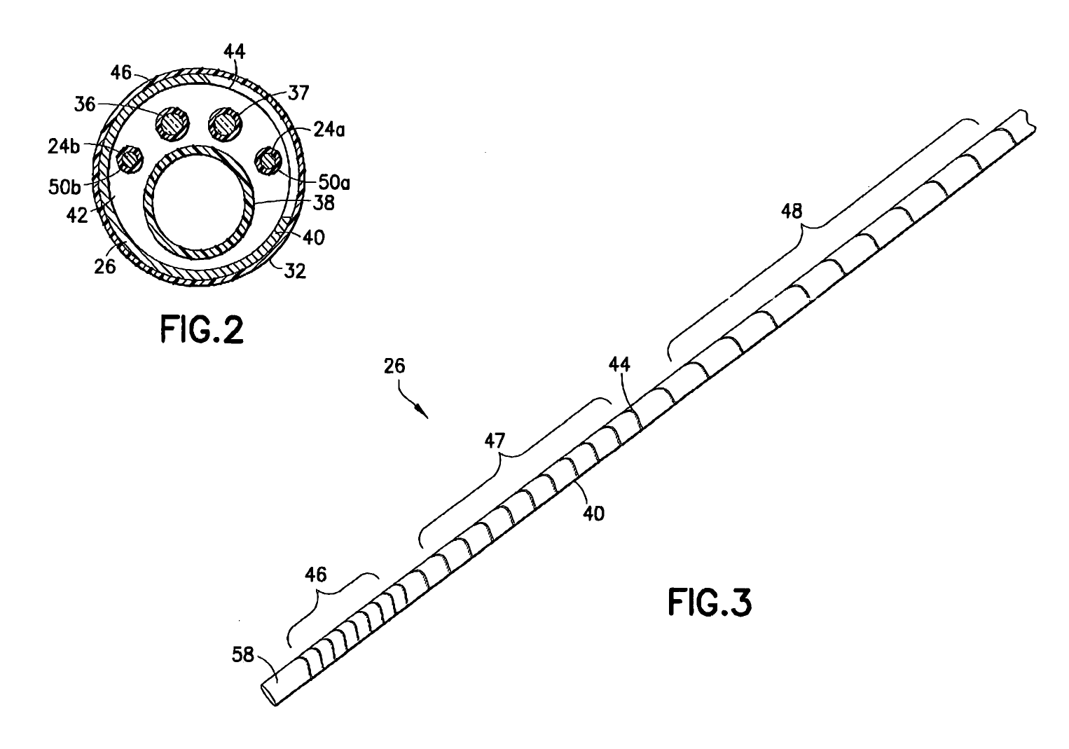

The endoscope 10 is a ureteroscope. However, in alternate embodiments the endoscope could be any suitable type of endoscope. The endoscope 10 generally comprises a handle or control 12 and a flexible or semi-flexible shaft 14 connected to the handle 12. The shaft 14 includes a passive deflection section 16 and an active deflection section 18 at the distal end of the shaft 14. A control system 22 to control the active deflection section 18 extends from the handle 12 to the active deflection section 18. Referring also to FIG. 2, the control system 22 generally comprises a pair of control wires 24a, 24b, two wire sheaths 50a, 50b, and an actuator 28. The wires 24a, 24b are connected to the actuator 28 at one end and are connected to the active deflection section 18 at a second end.

In the preferred embodiment, the handle 12 has a user operated slide or lever 30. The lever 30 is connected to the actuator 28. The actuator 28 is adapted to pull and release the two wires 24a, 24b of the control system 22. When the lever 30 is moved by the user, the actuator 28 is moved. The actuator 28 may be a drum or pulley rotatably connected to the handle 12 to pull one wire 24a, 24b while releasing the other. In an alternate embodiment, the actuator may be any suitable type of device, such as a rocker arm adapted to pull and release the wires of the control system 22. In another alternate embodiment, where the control system may have two or more pairs of control wires, the handle will have additional actuators and corresponding controls to drive the additional pairs of control wires. In still other alternate embodiments, the handle may have knobs with rack and pinion mechanisms or other suitable user operated controls for the control system.

The shaft 14 is cantilevered from the handle 12. The flexible shaft 14 includes the control wires 24a, 24b of the control system 22, a fiber optical image bundle 37, a fiber optical illumination bundle 36, and a working channel 38. Alternatively, rather than the fiber optical illumination bundle 36, the front of the shaft could comprise at least one light, such as at least one light emitting diode (LED), with an electrical wire extending through the shaft to the LED(s). A port 60 for inserting instruments (not shown) into the channel 38 is located on the handle 12. The handle 12 has an electrical cable 63 for connection to another device, such as a video monitor for example. In an alternate embodiment, instead of the cable 63, the endoscope could have an eyepiece. In alternate embodiments, the flexible shaft may house different systems within.

The shaft 14 generally comprises a frame 26, a cover 32 and an objective head 34. Referring also to FIG. 3, the frame 26 generally comprises a one-piece tube or spiral shaped frame member 40. However, in alternate embodiments the frame could be comprised of more than one frame member, such as multiple frame members connected in series, and could comprise additional members. The frame member 40 is preferably comprised of a shape memory alloy material, such as Tinel or Nitinol. The shape memory alloy material is used for its superelastic properties exhibited by the material's ability to deflect and resiliently return to its natural or predetermined position even when material strains approach 4%, or an order of magnitude greater than the typical yield strain of 0.4% giving rise to plastic deformation in common metals. Thus, the term “superelastic alloy” is used to denote this type of material. The wire sheaths 50a, 50b may also be comprised of this type of material such as disclosed in U.S. Pat. No. 5,938,588 which is hereby incorporated by reference in its entirety. In an alternate embodiment the tube might not be comprised of a superelastic alloy.

The frame member 40 has a center channel 42 with open front and rear ends, and a single slot 44. The slot 44 has a general helical shape along the length of the frame member 40. In one method of manufacturing the slot, the slot 44 is cut into a tube of material, such as metal or a superelastic alloy for example, by a laser. Use of a laser has been found to be the easiest method of forming the sinusoidal shape of the spiral cut. Methods of forming a non-straight spiral cut (or spiral cut with non-straight opposing sides) other than by use of a laser would be much more difficult. However, any suitable method for forming the frame member with the slot 44 could be provided in alternate embodiments. In this embodiment the general helical shape forms a general coil shape with different pitches at different sections, such as sections 46, 47 and 48 for example. However, in alternate embodiments more or less sections could be provided and more than one slot could be provided. In alternate embodiments the frame member 40 could have more or less than three sections of different slot patterns, such as only one or two for example. In addition, rather than abrupt transitions between sections of different slot patterns, the tube could be provided with gradual or intermixed slot transition zones between sections. In this embodiment the frame member 40 also has a section 58 which does not have the slot 44 therein.

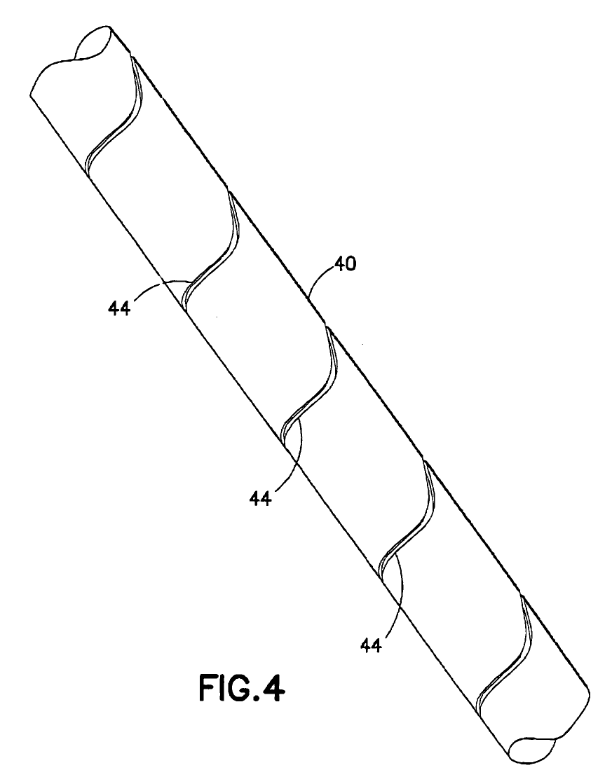

Referring also to FIGS. 4 and 5, enlarged views of one section of the frame member 40 is shown. As can be seen, the slot 44 has a general non-straight shape when viewed from the side. In this embodiment the gap between opposing surfaces 52, 54 which form the slot 44 is substantially uniform, but the surfaces 52, 54 themselves each have a general wavy or undulating shape. Thus, the slot 44 (although generally helical) also has a general wavelike or wavy pattern when viewed from the side. Stated another way, the surfaces 52, 54 have a general ripple shape or oscillating shape. The shape of the surfaces 52, 54 generally mirror each other, but could be different in different embodiments. In the hole position shown, the surfaces 52, 54 are spaced from each other.

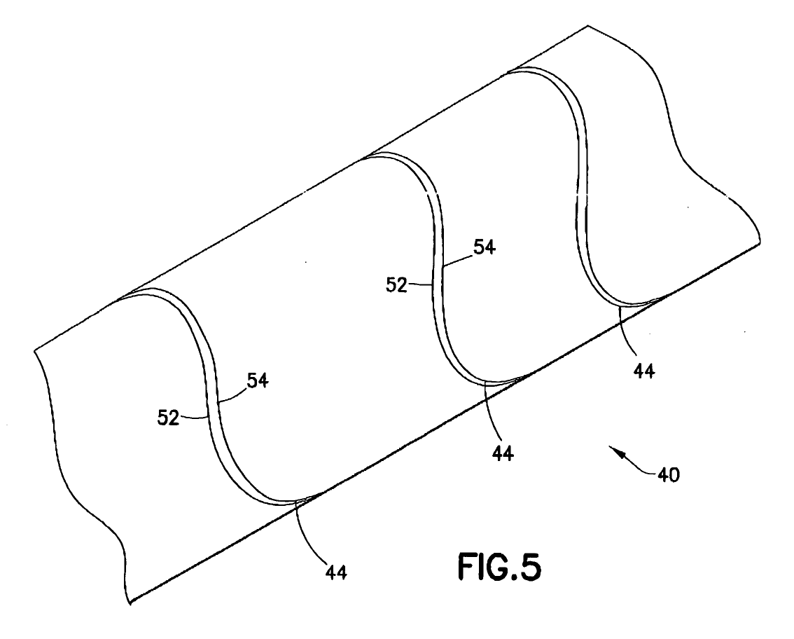

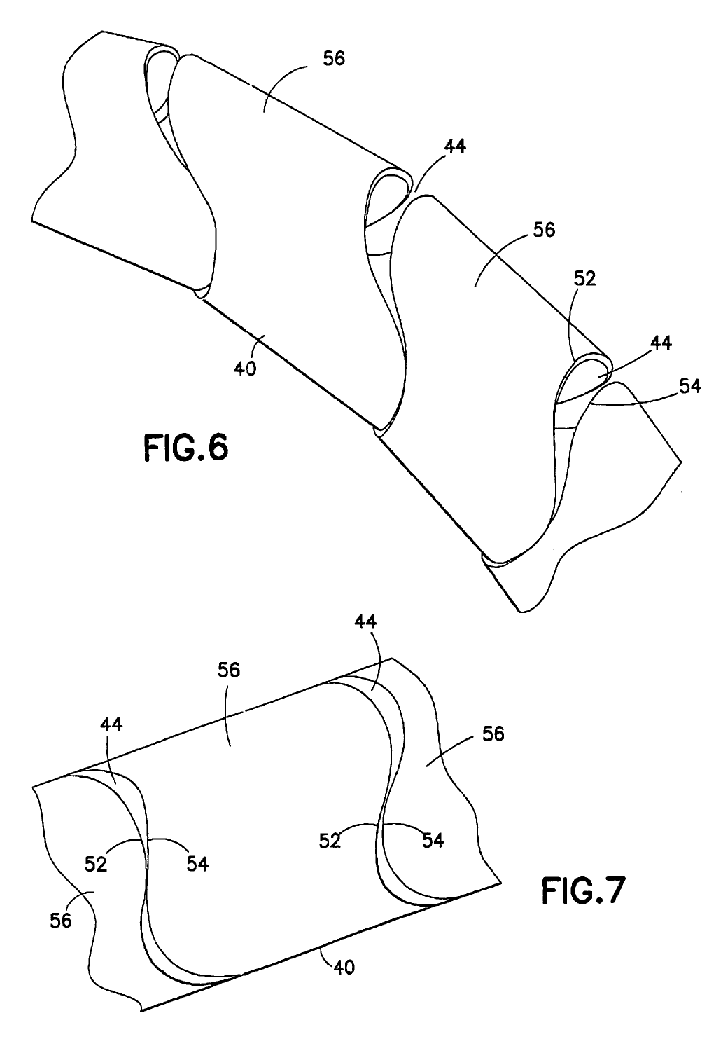

Referring also to FIG. 6, when the frame member 40 is bent, the slot 44 allows the coil portions 56 to move relative to each other. The surfaces 52, 54 can contact each other. Referring also to FIG. 7, when the frame member 40 is subjected to a torque force or twisting force, either left handed or right handed (clockwise or counter-clockwise), the wavy shape of the surfaces 52, 54 allows the opposing surfaces 52, 54 to contact one another and thereby limit axial rotation of the coil portions 56 relative to one another. Because of the shape of the surfaces 52, 54, it does not matter if the twisting force is clockwise or counter-clockwise; the surfaces 52, 54 will contact each other and limit deformation of the frame member 40. Thus, the frame member 40 can provide greater torque stability in a single member for both clockwise or counter-clockwise torque forces.

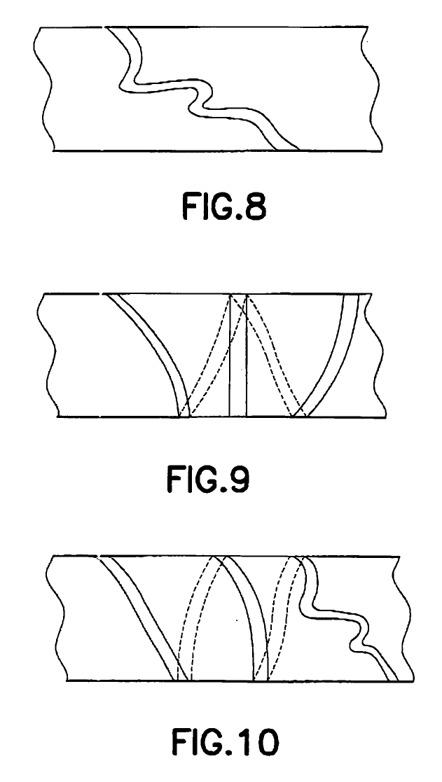

With the invention, the structure of the shaft can comprise a helical spiral or frame member made from resilient material. The spiral cut can be designed in a waved sinusoidal form. This feature can allow for reducing the risk of the shaft spiral being damaged from torque; in both left and right directions. With the invention, the shaft structure can have only a single spiral frame (compared with one of the conventional shafts which has two overlapping spiral frames), good resilient properties (compared to a conventional shaft which has an orthogonal cut shaft design), and excellent torque resistance in both right and left directions (without extra elements compared to a conventional shaft which has extra elements for torque stability). FIGS. 8-10 show some alternate embodiment illustrations to show that the slot(s) or portions of a slot do not need a uniform shape. The non-straight shape of the slot and/or the opposing surfaces on opposite sides of the slot might not be wavy and could vary.

With one embodiment of the invention, an endoscope shaft frame member 40 can be provided comprising a general tube shape with a section having a general helical shaped slot 44 along a length of the section, wherein the slot comprises a general wavy shape along a length of the slot. The frame member can comprise a shape memory alloy. The slot can have a general uniform width between opposing surfaces of the section at the slot. The opposing surfaces of the section at the slot can have a wavy pattern. The opposing surfaces of the section at the slot can undulate. The slot can form the section with a general uniform pitched coil shape.

With one embodiment of the invention, an endoscope shaft frame member 40 can be provided with a section comprising a general spiral shape having a general helical shaped slot 44 with a substantially uniform width between opposing surfaces 52, 54 of the section at the slot, wherein the slot is non-straight along its general helical shape at the section. The slot can undulate at the section. The slot can have a general wavy shape at the section. The frame member can comprise a shape memory alloy. The frame member can comprise metal. The opposing surfaces of the section at the slot can have a wavy pattern. The slot can form the section with a general uniform pitched coil shape.

In accordance with one embodiment of the invention, a method can be provided comprising providing a general tube shaped member; and forming a general helical shaped slot along a section of the tube shaped member, wherein the general helical slot 44 comprises a general wavy shape along its length. Forming the general helical slot at the section can comprise forming the slot with a general uniform width between opposing surfaces of the section at the slot. Forming the general helical slot at the section can comprise forming the slot with opposing surfaces of the section at the slot having a general wavy shape. Forming the general helical slot at the section can comprise forming the slot with opposing surfaces of the section at the slot having a general undulating shape. Forming the general helical slot at the section can comprise forming the slot with opposing surfaces of the section at the slot having a general ripple shape. Forming the general helical slot at the section can comprise forming the slot with opposing surfaces of the section at the slot each having a general oscillating frequency.

It should be understood that the foregoing description is only illustrative of the invention. Various alternatives and modifications can be devised by those skilled in the art without departing from the invention. For example, features recited in the various dependent claims could be combined with each other in any suitable combination(s). In addition, features from different embodiments described above could be selectively combined into a new embodiment. Accordingly, the invention is intended to embrace all such alternatives, modifications and variances which fall within the scope of the appended claims.

What is claimed is:

1. An endoscope shaft frame member comprising a general tube shape with a section having a generally helical shaped slot along a length of the section, wherein the slot comprises a general wavy shape along a length of the slot.

2. An endoscope shaft frame member as in claim 1 wherein the frame member comprises a shape memory alloy.

3. An endoscope shaft frame member as in claim 1 wherein the slot has a general uniform width between opposing surfaces of the section at the slot at the length of the slot.

4. An endoscope shaft frame member as in claim 1 wherein opposing surfaces of the section at the slot have a wavy pattern.

5. An endoscope shaft frame member as in claim 1 wherein opposing surfaces of the section at the slot undulate.

6. An endoscope shaft frame member as in claim 1 wherein the slot forms the section with a general uniform pitched coil shape.

7. An endoscope comprising:
a handle section; and
a shaft connected to the handle section, wherein the shaft comprises an endoscope shaft frame member as in claim 1.

8. An endoscope shaft frame member comprising a section having a general spiral shape formed by a generally helical shaped slot with a substantially uniform width between opposing surfaces of the section at the slot, wherein the slot is non-straight along its generally helical shape at the section.

9. An endoscope shaft frame member as in claim 8 wherein the slot undulates at the section.

10. An endoscope shaft frame member as in claim 8 wherein the slot has a general wavy shape at the section.

11. An endoscope shaft frame member as in claim 8 wherein the frame member comprises a shape memory alloy.

12. An endoscope shaft frame member as in claim 8 wherein the frame member comprises metal.

13. An endoscope shaft frame member as in claim 8 wherein the opposing surfaces of the section at the slot have a wavy pattern.

14. An endoscope shaft frame member as in claim 8 wherein the slot forms the section with a general uniform pitched coil shape.

15. A method comprising:
providing a general tube shaped member; and
forming a general helical shaped slot along a section of the tube shaped member, wherein the general helical slot comprises a general wavy shape along its length.

16. A method as in claim 15 wherein forming the general helical slot at the section comprises forming the slot with a general uniform width between opposing surfaces of the section at the slot.

17. A method as in claim 15 wherein forming the general helical slot at the section comprises forming the slot with opposing surfaces of the section at the slot having a general wavy shape.

18. A method as in claim 15 wherein forming the general helical slot at the section comprises forming the slot with opposing surfaces of the section at the slot having a general undulating shape.

19. A method as in claim 15 wherein forming the general helical slot at the section comprises forming the slot with opposing surfaces of the section at the slot having a general ripple shape.

20. A method as in claim 15 wherein forming the general helical slot at the section comprises forming the slot with opposing surfaces of the section at the slot each having a general oscillating frequency.
